(19) United States
(12) Patent Application Publication
(10) Pub. No.: US 2008/0280310 A1
(43) Pub. Date: Nov. 13, 2008

(54) Testing for Blood Group Immunological Reaction Without the Use of Anti-Human Globulin

(76) Inventor: Louis Panagopoulos, Niles, IL (US)

Correspondence Address: LOUIS PANAGOPOULOS, 8108 N. ORIOLE Ave., NILES, IL, 60714, US

(21) Appl. No.: 11/746,466

(22) Filed: May 9, 2007

(51) Int. Cl. G01N 33/53

(52) U.S. Cl. 435/7.92

(57) ABSTRACT

A method testing for blood antigens (known as forward blood grouping) is presented wherein known antibodies are attached to a solid surface and the red blood cell sample for immunological reaction is centrifuged to physically overcome the natural repellent force between two red cells (know in the industry as the zeta potential) and to allow for the antigen antibody reaction to occur more rapidly. A reverse blood grouping procedure utilizes synthetic or purified antigens, which are attached directly attached directly to a solid surface. The surface is then contacted with the patient's serum and then centrifuged to allow the antigen antibody reaction to occur. The cells are then washed and a second labeled antibody of known concentration is added as an indicator. This is a method of performing a major crossmatch that does not utilize anti-human immunoglobin. In an alternative major crossmatch procedure, a binder is used to attach red blood cell membranes from a blood donor and the serum from a recipient is allowed to undergo an immune reaction with these membranes on the solid surface. Antibody screening and antibody identification are carried out by attaching known antigen carrying cells to a solid surface. The solid surface is contacted with the unknown solution which will undergo an immune reaction to the extent that antibodies specific to the previously adhered antigens are present and will bind. Red blood cells or synthetic labeled particles are used as the indicator mechanism.

### FIELD OF THE INVENTION

This invention relates generally to immunohematology and, more particularly, to a method of determining the occurrence or non-occurrence of an immunological reaction between antigens and antibodies.

### BRIEF DESCRIPTION OF DRAWINGS

FIG. 1 is a top perspective view of a portion of a centrifuge with several wells attached thereto and several removed.

FIG. 2 is a top perspective view of a centrifuge with wells secured thereto.

FIG. 3 is also a top perspective view of a centrifuge with wells secured thereto.

### BACKGROUND OF THE INVENTION

The current state of the art of blood testing (blood typing) states that blood be tested for blood cell compatibility between a donor and recipient before a transfusion or the utilization of any other blood product. Blood cell compatibility is determined by whether or not there is an adverse an immunological reaction between the donor and the patient. An adverse immunological reaction is when antigens (which are markers contained on the blood of a patient) react with a preexisting antibody, or cause an antibody to be created in the blood serum of a patient. For example, a patient whose red blood cells are type “A” would have type “A” antigens in her red cells. If this patient is ever exposed to the blood group's conjugate antigen (in this case the “B” antigen) she will create anti-“N” antibodies in her serum. Thus, if such a person is given type “B” blood, an adverse immunological reaction will occur that could cause the patient to become compromised, ill, or to die. It is estimated that thousand of deaths occur each year due to adverse immunological reactions from transfusions and organ donation.

Blood group serology has most commonly been carried out by mixing blood and antibodies suspended in liquid and observing the immunological reaction. If certain components from two blood types (i.e. Antigen from one type and antibody to that antigen from another type) come into contact with each other, they form an adverse immunological interactions (called agglutination). This procedure is time consuming, labor intensive, and requires very skilled personnel. Furthermore, it only routinely detects a limited number of possible antigens and antibodies.

The method described in this patent is directed to a procedure for conducting blood testing in “solid phase.” Solid phase is specific term used to describe testing in which the material being tested is absorbed into solid surfaces. Solid phase testing is generally a simpler, less expensive, faster, and more reliable from of testing.

Centripetal force is used in this solid phase procedure to accelerate the testing process and significantly decrease the reaction time. It is this decreased reaction time that makes solid phase testing a more logical alternative to this type of clinical testing. Thus, with the addition of a chemical fixative, Anti-human globulin is no longer required for blood typing.

Previously, experts have believed that at is not feasible to make a solid phase blood-banking test without the use if anti-human globulin. It was taught in the prior art that a specific class of antibodies (known as IgG antibodies) could not bind strongly enough to antigens to allow them to be able to withstand centripetal force. For example, U.S. Pat. No. 4,608,246, Testing for a Blood Immunological Reaction, Bayer et. al. 1986 states:
Because Anti-D directly bound to a solid surface does not bind Rh positive red blood cells strongly enough to withstand centrifugal forces, it is desirable to bind Anti-D to anti-human immunoglobin (IgG) which has first been applied to the surface. Anti-human IgG is available from commercial sources and is incubated on the microtiter plates in a manner well known to those skilled in the art. Anti-D antibody, diluted in saline, is then added as discussed above for Anti-A and Anti-B.

In contrast to the teachings of the prior art, the method if the instant invention demonstrates that IgG antibodies can be directly attached to a solid matrix (a plastic or other device) for radio or enzyme linked immunoassays, and that the antigen antibody can be enhanced with a chemical fixative to allow it to be able to withstand centripetal force. The techniques previously used have been extremely effective, but never reached full potential because of their dependence on anti-human globulin.

### SUMMARY OF THE INVENTION

The present invention contemplates a method for testing ABO forward blood grouping pursuant to which a known antibody is attached directly to a solid surface with or without the help of a binding agent and tested. Pursuant to this method a patient blood sample is activated by treatment with an effective quantity of a proteolytic enzyme or other enhancing substances. The activated sample is then placed onto said solid surface and any antigens specific to the known antibody will undergo immune reaction. The wells will be centrifuged to accelerated the binding process and shorten the reaction time. The well is then to be washed to remove any unbound cells. The resulting adhered red color on the surface provides an indication of the presence of specific antigens.

The invention also comtemplates a method of testing applicable to reverse of serum ABO blood grouping. In this instance, a solid surface capable of supporting an immunological reaction is provided and known antigens are adsorbed onto the surface. The surface is then contacted with the patient's serum, which may contain unknown antibodies specific for the unknown antigen previously attached, in which case an immune reaction will occur. The sample well is to be centrifuged allowing the immunological reaction to occur more rapidly. Then the well is washed removing any unbound antibodies from the sample. Then a known concentration of labeled antibodies is added to the well, re-centrifuged, and read. The presence of labeled antibodies is significant.

Another aspect of the invention is a method of testing applicable to blood group or non-blood group immunological reactions wherein purified natural antigens or antibodies or synthetic antigens or antibodies are attached directly to a solid surface capable of supporting an immunological reaction. The surface is then contacted with a body fluid to permit an immune reaction between any antibody or antigen in the unknown fluid including, but not limited to blood, plasma, platelets, WBC's, cryoprecipitate, that is specific to an antigen or antibody previously attached to the solid phase. The well will be spun to allow an immunological reaction to occur. A solution comprising of a known concentration of labeled antibodies, or labeled antigens (depending on what is being tested for) is added to the well, re-centrifuged and read. The presence of any color is significant.

The invention does not include a method, of testing for a major blood group crossmatch or compatibility test by attaching anti-human immunoglobulin directly to a solid surface.

A method of antibody screening and antibody identification can also be carried out according to the present invention. A solid surface capable of adsorbing antigens is treated with a binding agent and then contacted with antibody screening cells carrying known antigens. The surface is then contacted with a blood component to allow any antibody in the component to react with any antigens previously attached to the surface. The well will be spun to allow an immunological reaction to occur. The well is then washed to remove unbound antibody. A solution comprising of a known concentration of labeled antibodies is added to the well, re-centrifuged, and read. The presence of any labeled antibody is significant.

### DETAILED DESCRIPTION OF EMBODIMENTS OF THE INVENTION

### Forward ABO Grouping and Rho (D) Typing

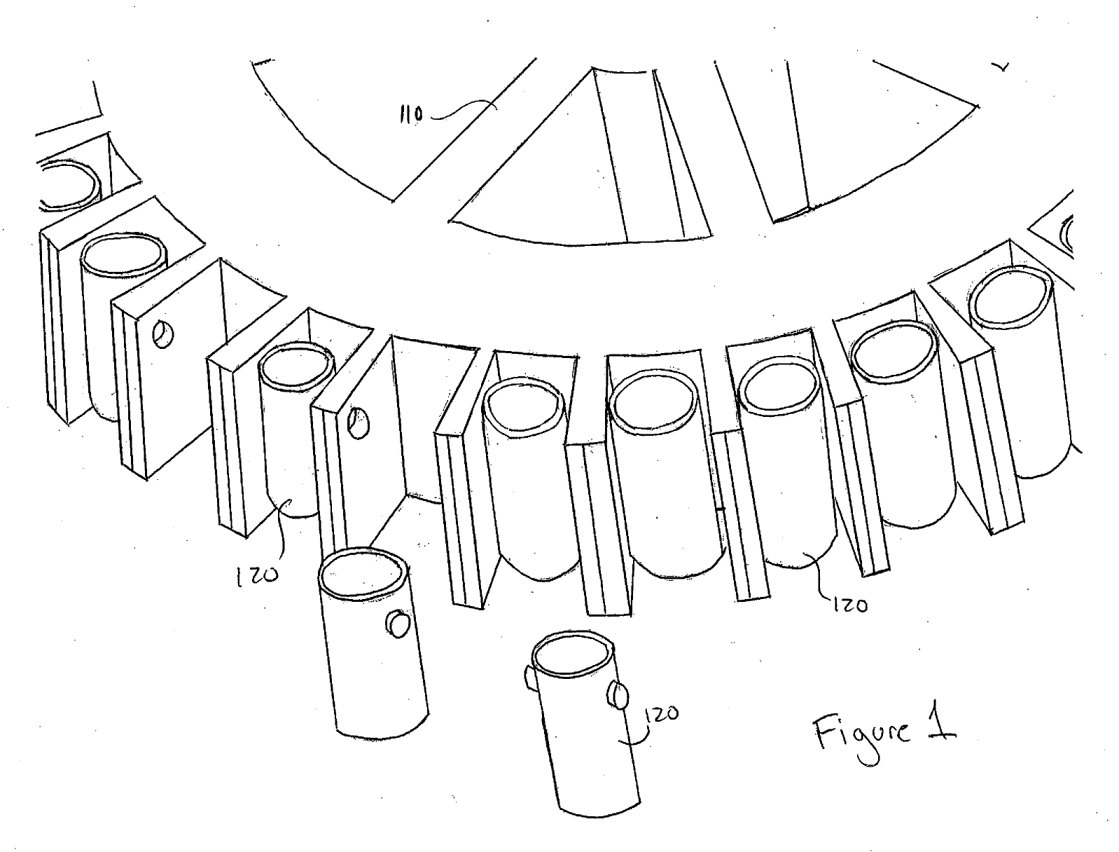

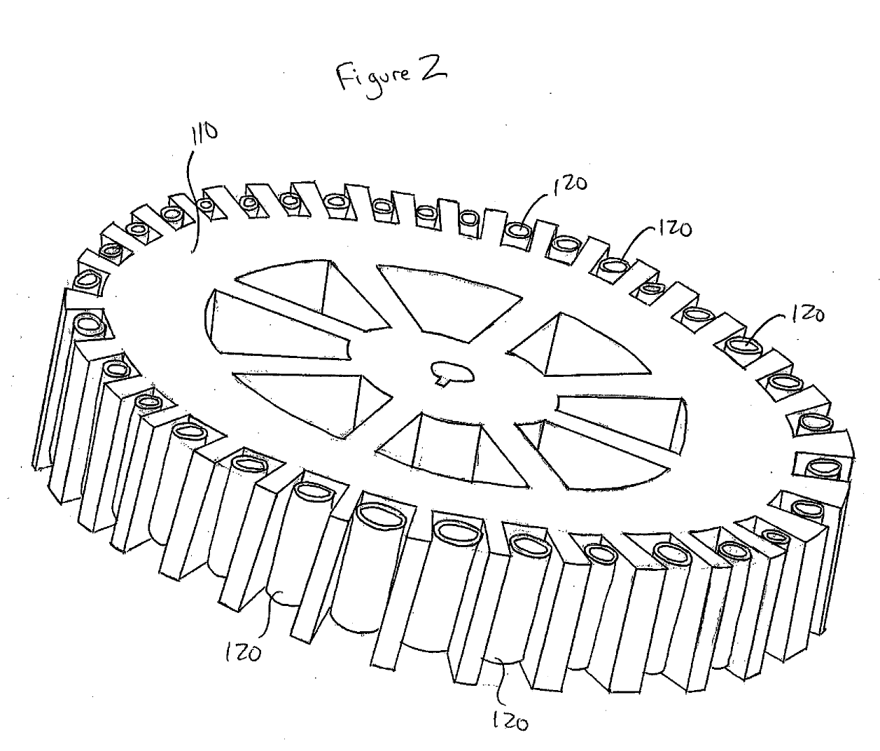

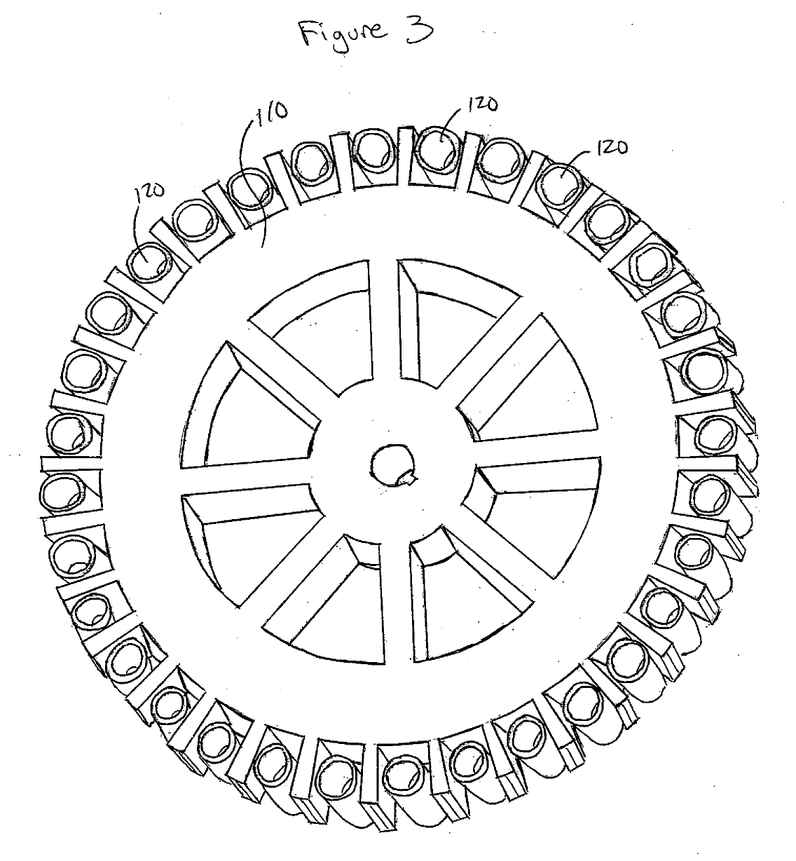

In one embodiment, the method of the present invention is carried out In solid phase on a solid surface capable of supporting an immunological reaction. Various materials provide adequate solid surfaces acceptable for the present method. Examples of acceptable substrates include beads, test tubes, sheets, strips, microtiter plates, and centrifuge heads 120 themselves (for use with centrifuge 110) such as the one shown In FIGS. 1, 2, and 3 all made of: polystyrene, polyvinylchloride, XPS, HIPS, SAM, ABS, PMMA, MBS, RPVC, CPVC, PB, LDPE, LLDPE, HDPE, HMWHDPE, LCP, PAS, PAEK, PC/ABS, PP/TALC, PP/GLASS, EVA, IN, CP, PEEK, PEI, PEKEKK, PES, POM, TPV, TPO, TP, PA6, PA66, PPA, PPE, PPS, PSO, PA11, PA12G, PA66M, PBT, PUR, TPI, PP, PP/CO, PET, PETG, PC, PVDF or any other applicable material. The wells may have flat bottom, U-shaped, or V-shaped bottoms. It is preferable to use a polystyrene centrifuge head, and wells having a U-shaped well, but not necessary.

The current art of attaching an antibody to a well states that; Anti-A or B antibody eluates are prepared from red blood cells or known commercial or noncommercial sources of synthetic A or B substances. A titer of at least 1:64 should be achieved for Anti-A and Anti-B eluates. Anti-D eluate is prepared in a similar manner although Anti-D is only attached and eluted from red blood cells. A liter of at least 1:64 should be achieved for Anti-D eluates. The techniques for preparing standardized solutions of Anti-A, Anti-B, and Anti-D are well known to those skied in the art. Alternatively, monoclonal anti-A, B or D antibodies or lectins can be used directly without further processing, it should be noted that the term “solution” as used throughout this specification is intended to encompass whole blood, red blood cell suspensions, serum and other blood components, body fluids, and mixtures unless otherwise noted.

The Anti-A and Anti-B solutions are diluted at least 1:20 with 60 millimolar (mM) Na.sub.2 CO.sub.3, pH 9.6. The diluted solutions are then added to the wells of the centrifuge head, about 100 microliters for a well of 325 microliter capacity. The Anti-A or Anti-B solution is incubated on the solid well surface for up to 18 hours at a temperature of 4.degree. C. or the time may be reduced to one hour if a temperature of about 37.degree. C. is maintained. Intermediate incubation times and temperature ranges are also acceptable. When the preferred wells having U-wells are employed, care should be taken to assure that the entire concave bottom walls of the wells are adequately coated.

Anti-D can be bound directly to a solid surface using a binding agent. If Anti-D Is treated with a paraformaldehyde solution, or any other applicable chemical fixative, it does bind Rh positive red blood cells strongly enough to withstand centrifugal forces. Anti-D is incubated on the microtiter plates in a manner well known to those skilled In the art, Anti-D antibody, diluted in saline, is then added as discussed above for Anti-A and Anti-B

It has been discovered that glutaraldehyde treatment, which has previously been known as a preservative and fixative, will enhance the adherence of the antibody-coated plates. Thus, to prolong shelf life of the antibody coated solid surface or enhance the adherence, a dilute glutaraldehyde solution is applied. A 25 weight percent biological grade glutaraldehyde solution is diluted to 2% with 10 mM NaH.suh.2 PO.sufo.4 and 140 mM NaCl; final pH should be about 5. A quantity of about 100 microliters per 325 microliters capacity well is added and Incubated for about 2 hours at room temperature. Anti-A, Anti-B or Anti-IgG/Anti-D antibody is then added as discussed above.

In carrying out the method of the present invention, it has been found that considerable advantages result when, in forward blood grouping, the blood being tested is activated with a proteolytic enzyme which is characterized by the ability to modify red blood cells to enhance their serological activity. Examples of suitable proteolytic enzymes for this purpose include Bromelain, Papain, Trypsin Ficin, Proteinase K (protease from Tritirachium album), and Pronase (protease from Streptomyces griseus). A one-step bromelain method is preferred. A 1% (by weight) stock solution of bromelain in phosphate buffered saline is diluted in additional phosphate buffered saline (pH 7.4) to a final concentration of 0.1%. This has been found to be an effective concentration, though not critical. Some results will be observed at concentrations of 0.05% or less, and generally a concentration of greater than 0.5% should be avoided.

Either whole blood or red blood cells at a concentration of 0.5% by volume for red blood cells and 1% by volume for whole blood are then subjected to the bromelain. Again, the concentration levels are not critical and could vary by from one-fifth to five times the amounts stated. Preferably, the whole blood or red blood cells are incubated with the bromelain for 1 to 15 minutes at room temperature.

The bromelain or other enzyme activated blood component (this term encompassing both whole blood and red blood cells) is then added to the individual wells of the previously prepared antibody coated microtiter plates. The blood component can be allowed to settle in the wells unaided although it is preferable to centrifuge for about 30 seconds at 150 times G. Any blood group antigen present in the blood component that is specific for the antibody attached to the well will undergo an immune reaction and adhere to the bottom of the well. A positive reaction is indicated by a uniform effacement of red blood cells over the entire concave bottom surface of the well. After the red blood cells have been allowed to adhere, the solid surface is washed with 0.85 weight percent NaCl to remove any unreacted and thus unbound red blood cells. A negative reaction will be characterized by the absence of red blood cells. Alternatively, the well can be left as is, a negative reaction will be characterized by all of the red blood cells settling to the bottom of the well into a small “button” in the center. Another alternative is to invert the plate and centrifuge for 30 seconds to remove any unbound red blood cells. In these latter two cases, the negative reaction will be indicated by a clear well devoid of red blood cells. The plates may be read visually or mechanically with a densitometer which can be interfaced to a computer for data interpretation and processing.

All of the aforedescribed techniques are applicable to the subsequently described procedures unless noted contra.

### Reverse Blood Grouping

The method of the present invention is applicable to reverse ABO blood grouping where known antigens are attached to a solid surface of the type previously specified to determine the presence of specific antibodies. Again, plastic centrifuge heads having U-shaped wells are the preferred solid surface. In this application, it has been found particularly advantageous to utilize synthetic A or B substances or purified antigens obtained from either human red blood cell membranes, secretions such as human saliva, or animal tissues such as equine and porcine stomach. These synthetic or purified substances have the advantage that they can be attached directly to the solid surface in the manner previously described for Anti-A and Anti-B antibodies. This results in significant time savings and the synthetic or purified antigens offer greater sensitivity and specificity as well.

In the event that whole blood or a suspension of red blood cells is utilized for the reverse grouping procedure, it is necessary to coat the microtiter plates with a binder such as plant or animal lectins. Suitable substances include Canavalia ensiformis, Lens culinaris, Pisum sativum, Triticum vulgaris, Glycine max, Limulus polyphemus, Helix pomatia, Helix aspersa, and Phaseolus limensis. The technique for applying the foregoing binders to solid surfaces is well known.

In performing reverse grouping according to the method of the present invention, if whole blood or a suspension of red blood cells are used as the known antigen source, it has been found particularly advantageous to treat the blood component with a proteolytic enzyme as discussed previously in conjunction with forward grouping techniques. Also, when red blood cells or whole blood is used, it is necessary to lyse the cells to remove hemoglobin. This provides a clear background for the detection of positive or negative immune reactions.

The prepared solid surface with antigens attached may be glutaraldehyde fixed in the manner in which the substrates with antibodies attached are glutaraldehyde fixed as discussed above. It is to be noted, however, that with an antigen carrying surface, glutaraldehyde fixation is carried out subsequent to attachment.

Either serum or whole blood may be used for reverse grouping and a sample of the unknown component (this term encompassing both serum, whole blood, body fluid, etc.) is brought into contact with the previously prepared solid surface to allow any antibodies present to bind to the antigens fixed to the solid surface. The unknown blood component is cenfrifuged to allow the antigen antibody reaction to occur. Next, non-specific antibodies are removed, preferably by washing the surface with saline solution. Any blood group antibody specific for the antigen attached to the plate will undergo an immune reaction and adhere to the solid surface. An additional immune reaction is required to indicate the presence of the specific antibodies. For this purpose, a known solution of labeled antibodies is added to the well and the well is re centrifuged to create an antigen antibody reaction. The well is washed once more with saline to remove any unbound antibody, and the result is read. The method of reading is dependent on the label present on the antibody.

While the method of the instant invention has been shown and described with respect to several embodiments in accordance with the present invention, it is to be understood that the same is not limited thereto, but is susceptible to numerous changes and modifications as known to a person skilled in the art, and it is intended that the present invention not be limited to the details shown and described herein, but rather cover all such changes and modifications obvious to one of ordinary skill in the art.

We claim:

1. A method of testing for a blood group antigen-antibody immunological reaction without the use of Anti-human globulin said method comprising:
providing a solid surface capable of adsorbing blood grouping antibodies and supporting an immunological reaction;
contacting said surface with a known blood grouping antibody containing fluid whereby a layer of said antibodies is adsorbed on said surface;
activating an unknown blood sample by treating said sample with an effective quantity of a proteolytic enzyme characterized by the ability to modify red blood cells to enhance their serological activity; and
contacting said surface with said activated blood sample whereby any antigen specific to said antibody will undergo an immune reaction with said antibody and the resulting immunologically adhered red color on said surface will indicate the presence of specific antigens in said unknown blood sample.

2. A method as set forth in claim 1, wherein said step of providing a solid surface comprises providing a plate, or centrifuge head having a well and said contacting steps comprise contacting the bottom wall, or sides of said well.

3. A method as set forth in claim 1, wherein said activating step comprises treating said sample with an effective quantify of a proteolytic enzyme selected from the group consisting of Bromelain, Papain, Trypsin, Ficin, Proteinase K, and Pronase, or any other proteolytic enzyme.

4. A method as set forth in claim 1, wherein said contacting step comprises centrifugation of the sample wells.

5. A method as set forth in claim 1, wherein said known antibody containing fluid comprises Anti-D and said contacting step comprises contacting said surface with anti-D substance alone (the same is true for any other antibody to be tested for).

6. A method as set forth in claim 1, wherein said method includes, subsequent to said first contacting step, the step of adding to said surface a paraformaldehyde, or any other applicable fixative solution to ensure the strength of the IgG antigen antibody bond.

7. A method of testing for a blood group immunological reaction, said method comprising:
providing a solid surface capable of adsorbing antigens and supporting an immunological reaction;
treating said surface with a binding agent;
providing a quantify of known antibodies for contact with surface;
contacting said surface with said activated known antibodies;
contacting said surface with the sample of red blood cells to be tested whereby any antigen in said sample specific to said antibody in said well will undergo an immune reaction; and
washing said surface to remove any unbound red blood cells, and any resulting immunologically adhered red color on said surface will indicate the presence of specific antibodies in said unknown blood component.

8. A method as set forth in claim 7, wherein said step of treating said surface with a binding agent comprises treating said surface with a member of the group consisting of plant and animal lectins.

9. A method as set forth in claim 7, wherein said activating step comprises treating said sample with an effective quantity of a proteolytic enzyme selected from the group consisting of Bromelain, Papain, Trypsin, Ficin, Proteinase K, and Pronase or any other proteolytic enzyme.

10. A method of testing for an antigen-antibody immunological reaction without the use of Anti-human globulin said method comprising:
providing a solid surface capable of adsorbing antibodies and supporting an Immunological reaction:
contacting said surface with a known antibody containing fluid whereby a layer of said antibodies is adsorbed on said surface;
activating an unknown blood sample by treating said sample with an effective quantify of a proteolytic enzyme characterized by the ability to modify antigen sites to enhance their serological activity; and
contacting said surface with said activated sample whereby any antigen specific to said antibody will undergo an immune reaction with said antibody and the resulting immunologically adhered color on said surface will indicate the presence of specific antigens in said unknown blood sample.

11. A method as set forth in claim 10, wherein said step of providing a solid surface comprises providing a plate, or centrifuge head having a well and said contacting steps comprise contacting the bottom wall, or sides of said well.

12. A method as set forth in claim 10, wherein said activating step comprises treating said sample with an effective quantity of a proteolytic enzyme selected from the group consisting of Bromelain, Papain, Trypsin, Ficin, Proteinase K, and Pronase, or any other proteolytic enzyme.

13. A method as set forth in claim 10, wherein said contacting step comprises centrifugation of the sample wells.

14. A method as set forth in claim 10, wherein said known antibody containing fluid comprises Anti-D and said contacting step comprises contacting said surface with anti-D substance alone (the same is true for any other antibody to be tested for).

15. A method as set forth in claim 10, wherein said method includes, subsequent to said first contacting step, the step of adding to said surface a paraformaldehyde, or any other applicable fixative solution to ensure the strength of the IgG antigen antibody bond.

16. A method as set forth in claim 10, wherein said antigen-antibody immunological reaction without the use of Anti-human globulin said method comprising of antigen groups Including but not limited to those on platelets, cluster designation groups on white blood cells, HLA antigens, and all comparable veterinary applications

17. A method of testing for an immunological reaction, said method comprising:
providing a solid surface capable of adsorbing antigens and supporting an immunological reaction;
treating said surface with a binding agent;
providing a quantity of known antibodies for contact with surface;
contacting said surface with said activated known antibodies;
contacting said surface with the sample of red blood cells to be tested whereby any antigen in said sample specific to said antibody in said well will undergo an immune reaction; and
washing said surface to remove any unbound red blood eels, and any resulting immunologically adhered red color on said surface will indicate the presence of specific antibodies in said unknown blood component.

18. A method as set forth in claim 17, wherein said step of treating said surface with a binding agent comprises treating said surface with a member of the group consisting of plant and animal lectins.

19. A method as set forth in claim 17, wherein said activating step comprises treating said sample with an effective quantity of a proteolytic enzyme selected from the group consisting of Bromelain, Papain, Trypsin, Ficin, Proteinase K, and Pronase or any other proteolytic enzyme.
